(19) United States
(12) Patent Application Publication
(10) Pub. No.: US 2026/0053848 A1
(43) Pub. Date: Feb. 26, 2026

(54) ORAL FORMULATIONS COMPRISING POROUS SILICA PARTICLES AND MEDICAL USES THEREOF

(71) Applicant: Sigrid Therapeutics AB, Stockholm (SE)

(72) Inventors: Melissa Louise Borg, Gustavsberg (SE); Ghislaine Monique Nicole Robert-Nicoud, Stockholm (SE); Jeanha Baek, Stockholm (SE); Eric Johnston, Bromma (SE); Tore Bengtsson, Vaxholm (SE)

(21) Appl. No.: 19/105,311

(22) PCT Filed: Aug. 23, 2023
(86) PCT No.: PCT/EP2023/073128

§ 371 Date: Feb. 20, 2025

(30) Foreign Application Priority Data
Aug. 23, 2022 (GB) 2212240.2

(51) Int. Cl. A61K 33/00; A61K 9/00; A61K 47/36; A61K 47/38

(52) U.S. Cl. CPC A61K 33/00; A61K 9/0053; A61K 47/36; A61K 47/38

(57) ABSTRACT

There is provided herein an oral formulation comprising: (a) water; (b) porous silica particles having pores in the mesoporous range; and (c) a thickening agent, wherein the average pore size of the pores in the mesoporous range is from about 7.0 to about 25.0 nm; and the thickening agent is selected from xanthan gum and microcrystalline cellulose or a mixture thereof. There are also provided uses of such formulations.

### FIELD OF THE INVENTION

The present invention relates to novel formulations and uses of such formulations, such as their use in medicine.

In particular, the present invention relates to novel aqueous formulations, comprising silica particles and a thickening agent, for oral administration, and to the use of such formulations in methods for the treatment or prophylaxis of metabolic diseases and disorders, such as prediabetes, type 2 diabetes, dyslipidaemia and obesity. It also relates to the use of such formulations in methods for delaying digestion of food and reducing uptake of biomolecules into the body from the digestive system.

### BACKGROUND OF THE INVENTION

The listing or discussion of any prior-published document in this specification should not necessarily be taken as an acknowledgement that the document is part of the state of the art or is common general knowledge.

Metabolism is the sum of all energetic processes in the body and metabolic disorders disrupt normal metabolism, including the process of converting food to energy. A metabolic disorder occurs when abnormal chemical reactions in the body disrupt the process of metabolism. There are different groups of disorders. Some affect the breakdown of amino acids, carbohydrates, or lipids. Another group, mitochondrial diseases, affects the parts of the cells that produce the energy. Metabolic disorders can be categorised as either primary (genetic) or secondary (relating to lifestyle and environment among others).

Metabolic syndrome is a medical term that defines a clustering of at least three of the five following medical conditions: abdominal obesity, high blood pressure, high blood sugar, high serum triglycerides and low high-density lipoprotein (HDL) levels, such that the conditions occur together. Metabolic syndrome is indicative of an increased risk of cardiovascular diseases and type 2 diabetes.

Diabetes is the most common metabolic disease. There are two main types of diabetes: type 1 and type 2. Both types of diabetes are chronic diseases that affect the way the body regulates blood sugar (glucose). There is a strong genetic (hereditary) factor involved in the development of type 1 diabetes. There are several causes of type 2 diabetes, including genetics and lifestyle choices.

Prior to developing type 2 diabetes, patients suffer from a medical condition called prediabetes. Prediabetes is characterized by elevated blood sugar (glucose) levels above what is considered normal, but not high enough to be classified as type 2 diabetes. Prediabetes is a high-risk state for developing type 2 diabetes. If left untreated, 15-30% of people with prediabetes will develop type 2 diabetes within five years (U.S. Department of Health and Human Services, Centers for Disease Control and Prevention (2014)). The number of individuals with prediabetes is expected to grow substantially and estimated to globally affect 482 million people by 2040.

There are currently no approved medical treatments in the guidelines for prediabetes. The standard of care for prediabetes management is lifestyle intervention. Prediabetic patients are managed by primary care where they are recommended to exercise and eat healthily. Lifestyle intervention has a low compliance rate, with only 10-20% of patients able to implement a new lifestyle. Thus, there is currently a major unmet clinical need for treatment of prediabetes, with only 3.7% of prediabetics currently considered to be suitable for treatment with metformin (see Moin, T. et al., Ann. Intern. Med., 162, 542 (2015)).

According to the US Centers for Disease Control and Prevention, rates of type 2 diabetes have tripled in the past 30 years. This is caused largely by the global epidemic of obesity, a major risk factor for developing type 2 diabetes and prediabetes. Obesity is defined as abnormal or excessive fat accumulation that may impair health. At a fundamental level, obesity occurs when, over time, the body takes in more calories than it burns. Obesity increases the risk of developing a number of chronic diseases, including: insulin resistance, type 2 diabetes, high blood pressure, high cholesterol, stroke, heart attacks, sleep apnea, congestive heart failure, osteoarthritis and cancer. In particular, high levels of cholesterol and lipids (lipid disorders) have been associated with cardiovascular diseases as well as atherosclerosis.

Dyslipidaemia is regarded as a metabolic disease, being defined as an abnormal amount of lipids (e.g. cholesterol and/or fat) in the blood. This is often due to diet and lifestyle. Cardiovascular disease is often grouped with metabolic disorders because it is frequently a consequence of diabetes and dyslipidaemia.

Statins are the most widely used lipid lowering drug for prevention of coronary diseases in high risk patients. There are however controversies regarding their positive effects in preventing death and cardiovascular diseases in low and moderate risk patients (Tonelli, M., CMAJ, 183, 1189-1202 (2011); Ward, S., Health Technol Assess., 1-160 (2007)). Furthermore, there is a large number of intolerant patients who experience adverse events, such as liver damage, neurological effects and muscle pains (see, for example, Silva M. A. et al., Clin Ther., 28(1), 26-35 (2006) and Beatrice A. G., et al., American Journal of Cardiovascular Drugs, 8(6), 373-418 (2008)). The latter is the major side effect limiting the use of statins (Martini, 3. et al., Canadian J of Cardiology, 27, 635-662 (2011)). Hence, there is a need for new and more efficient lipid lowering treatment alternatives with and without the combination of lipid lowering drugs.

In view of the above, it is clear that there is a major drive for the development of new initiatives, medicines and medical technologies that offer effective, fast-acting and safer prevention and treatment methods to delay and treat prediabetes, diabetes and their related diseases.

It has previously been shown that silica particles with specific porosity can lower adipose tissue in animal model systems (see: WO 2014/072363 and Kupferschmidt, N. et al., Nanomedicine, 9(9), 1353-1362 (2014)). Animals receiving large pore mesoporous silica with a high-fat diet showed a significant reduction in body weight and body fat composition, with no observable negative effects. In particular, the authors propose the use of such silica for reduction of body weight and body fat composition as a means for the treatment of obesity.

It has been reported that such silica materials may also reduce levels of HbA1c, which is a biomarker the levels of which correspond to long-term plasma glucose levels (see Baek, J. et al., Nanomedicine, 17:1, 9-22 (2022)).

Furthermore, it has been shown that the porosity of such silica particles can be adjusted to achieve different physiochemical properties (see WO 2019/166656).

Porous silica particles are thermally and chemically stable, and are exclusively composed of pure silicon dioxide. They have controllable pore dimensions, which can provide high surface areas and large total pore volume. These properties, amongst others such as stability and biocompatibility, make them suitable for biomedical applications (Wang, Y. et al., Nanomedicine Nanotechnology, Biol. Med., 11, 313-327 (2015)). Moreover, similar materials have previously been approved as food additives (European Center for Ecotoxicology and Toxicology of Chemicals Synthetic Amorphous Silica (CAS No. 7631-86-9), JACC No. 51, page 14 (ECETOC, 2006)).

However, the use such silica particles in methods of medical treatment poses significant challenges, particularly as such particles are typically to be administered orally.

The dose of silica material required in order to achieve a therapeutic effect is often relatively large, typically in the region of several grams, which renders the presentation of such material in the form of tablets or capsules inconvenient, as limitations on the size and shape of individual tablets and capsules suitable for oral administration would result in a need for the consumption of multiple dosage units.

In order to address this problem, silica material may be presented in the form of an orally consumable liquid. However, such liquid formulations suffer from instability, in that the silica material may separate from the formulation such that it becomes unevenly distributed, and a poor taste profile, such as having a gritty texture and inducing feelings of dry mouth following consumption.

There exists, therefore, a need for oral formulations of silica material that are stable and do not inhibit the function of the silica material, so that they would be suitable for uses such as those described herein. Ideally, such formulations will also have a pleasant texture, such that they are more readily consumed.

### DETAILED DESCRIPTION OF THE INVENTION

It has unexpectedly been found that improved oral formulations of therapeutically active silica material may be prepared through the addition of certain thickening agents. Such formulations may be of use as dietary supplements and as pharmaceutical agents or medical devices, which may be of use in treating diseases and disorders as described herein.

### Novel Formulations

In a first aspect of the invention, there is provided an oral formulation comprising:
(a) water;
(b) porous silica particles having pores in the mesoporous range; and
(c) a thickening agent,

wherein:

the average pore size of the pores in the mesoporous range is from about 7.0 to about 25.0 nm; and

the thickening agent is selected from xanthan gum and microcrystalline cellulose or a mixture thereof.

Unless indicated otherwise, all technical and scientific terms used herein will have their common meaning as understood by one of ordinary skill in the art to which this invention pertains.

For the avoidance of doubt, formulations as defined in the first aspect of the invention (including all embodiments and features thereof) may also be referred to as “the formulation(s) of the invention (or of the first aspect of the invention),” or the like. Such formulations may also be referred to as compositions, which terms may be used interchangeably. Similarly, the silica particles as defined in the first aspect of the invention (including all embodiments and features thereof) may also be referred to as “the silica (or silica material or silica particles) of the invention (or of the first aspect of the invention),” or the like.

When used herein in relation to a specific value (such as an amount), the term “about” (or similar terms, such as “approximately”) will be understood as indicating that such values may vary by up to 10% (particularly, up to 5%, such as up to 4%, 3%, 2% or 1%) of the value defined. It is contemplated that, at each instance, such terms may be replaced with the notation “±10%”, or the like (or by indicating a variance of a specific amount calculated based on the relevant value). In relation to percentage amounts referred to herein, the term “about” (or similar terms, such as “approximately”) will be understood as indicating that such values may vary by up to 10% (particularly, up to 5%, such as up to 4%, 3%, 2% or 1%) of the percentage value defined. It is also contemplated that, at each instance, the term about may be deleted.

For the avoidance of doubt, references to formulations comprising certain components will include the possibility that such formulations may consist of (or consist substantially of) those components.

For the avoidance of doubt, the skilled person will understand that where percentages of a certain component are defined as belonging to different (i.e. non-overlapping) groups, the sum of those percentages cannot exceed 100%. Thus, where the maximum values of one or more non-overlapping groups of components would exceed 100%, the skilled person will understand that one or more of those components must be present in an amount that is less than the maximum value. Similarly, where it is possible for other components to be present in a formulation, there is no requirement for the sum of the specified components to equal 100%.

The skilled person will understand that references to an oral formulation will refer to a formulation for (e.g. suitable for) oral consumption, such as oral consumption by a human or animal (e.g. a mammal, such as a household pet or recreational animal, such as a cat, dog, horse, rabbit, or the like, or livestock, such as a cattle, pigs, sheep, goats, chickens, turkeys, or the like). In certain instances, references to consumption by such human or animal subject (which, in relation to methods of medical treatment, may also be referred to as patients), will refer in particular to treatment of adult (e.g. fully grown) subjects.

In particular, references to an oral formulation will refer to a formulation for (e.g. suitable for) oral consumption by a human, such as an adult human (e.g. a human of at least 18 years of age).

The skilled person will understand that references herein to oral consumption will refer to the formulation being swallowed, either in one action or several, by the subject. As such, the skilled person will understand that the formulation of the invention may be in the form of a liquid, gel or soft solid, such that the formulation may be easily swallowed.

The skilled person will understand that it may be advantageous for the formulation to be provided in the form of a readily flowable liquid, such that it may be consumed by the subject in the manner of a drink.

Thus, in particular embodiments, the formulation is a liquid (in which case it may be referred to as a liquid formulation).

The skilled person will understand that the term liquid will typically refer to a substance that flows freely but is of constant volume, typically having a consistency similar to that of water or a liquid oil.

In further embodiments, the formulation may be provided in the form of a soft solid/gel, which may have a consistency similar to that of a yogurt (e.g. a set yogurt, which may be described as a spoonable consistency).

The skilled person will understand that the amount of each component present in the formulation may be adjusted in order to achieve the required viscosity and flowability.

In particular embodiments, the formulation may be provided in a form having a viscosity classified as low to medium, such as having a viscosity and/or flowability similar to that of a milkshake or similar beverage/food item.

In further embodiments, the formulation may be provided in a form having a viscosity classified as high, such as having a viscosity and/or flowability similar to that of a set yogurt or similar food item.

As described herein, the formulation of the invention comprises water. In particular, the formulation will typically comprise water as a significant component, such as being the major (i.e. largest) component by weight. The formulation may therefore be described as being an aqueous (or substantially aqueous) formulation.

The skilled person will understand that the amount of water included in the formulation may be selected in order to achieve the desired properties of the formulation, such as the desired viscosity and/or flowability.

In particular embodiments, the formulation comprises:
at least about 70.0% (such as at least about 71.0, 72.0, 73.0, 74.0, 75.0, 76.0, 77.0, 78.0, 79.0%) by weight (wt %) of water; and/or (e.g. and) at least about 80.0 wt % (such as at least about 81.0, 82.0, 83.0, 84.0 or 85.0 wt %) water.

In particular embodiments, the formulation comprises up to about 95.0 wt % (such as up to about 94.0, 93.0, 92.0 or 91.0 wt %) water.

In more particular embodiments, the formulation comprises up to about 90.0 wt % (such as up to about 89.0, 88.0, 87.0, 86.0 or 85.0 wt %) water.

For example, in particular embodiments, the formulation comprises from about 70.0 to about 95.0 wt % water.

In more particular embodiments, the formulation comprises from about 75.0 to about 95.0 wt % water.

In more particular embodiments, the formulation comprises from about 80.0 to about 95.0 wt % water.

In more particular embodiments, the formulation comprises from about 75.0 to about 90.0 wt % water.

In more particular embodiments, the formulation comprises from about 80.0 to about 90.0 wt % water.

In more particular embodiments, the formulation comprises from about 82.0 to about 88.0 wt % water.

In more particular embodiments, the formulation comprises from about 83.0 to about 87.0 wt % water.

In more particular embodiments, the formulation comprises from about 84.0 to about 86.0 wt % water.

In a certain embodiment, the formulation comprises from about 84.0 to about 91.0 wt % water.

In a certain embodiment, the formulation comprises from about 84.0 to about 90.0 wt % water.

In a particular embodiment, the formulation comprises about 85 to about 86 wt % water, such as about 85.6 wt % water.

In a particular embodiment, the formulation comprises about 89 to about 90% wt % water, such as about 89.0 to about 90.0% wt % water.

In certain embodiments, it may be understood that, where the amounts of other ingredients are specified, the remainder of the composition (e.g. by weight) is water.

As described herein, the formulation of the invention comprises porous silica particles having pores in the mesoporous range, wherein the average pore size of the pores in the mesoporous range is from about 7.0 to about 25.0 nm.

The skilled person will understand that the amount of such porous silica particles in the formulation may be selected in order to provide an appropriate loading such that administration of a suitable amount of the formulation delivers the desired dose of silica material (e.g. the dose required in order to achieve the desired biological effect, such as the therapeutic and non-therapeutic effects described herein).

In particular embodiments, the formulation comprises:
at least about 5.0 wt % (such as at least about 6.0, 7.0, 8.0 or 9.0 wt %) of the porous silica particles; and/or (e.g. and)
up to about 15.0 wt % (such as at least about 14.0, 13.0, 12.0 or 11.0 wt %) of the porous silica particles.

In particular embodiments, the formulation comprises from about 5.0 to about 15.0 wt % of the porous silica particles.

In more particular embodiments, the formulation comprises from about 6.0 to about 14.0 wt % of the porous silica particles.

In more particular embodiments, the formulation comprises from about 7.0 to about 13.0 wt % of the porous silica particles.

In more particular embodiments, the formulation comprises from about 8.0 to about 12.0 wt % of the porous silica particles.

In more particular embodiments, the formulation comprises from about 9.0 to about 11.0 wt % of the porous silica particles.

In more particular embodiments, the formulation comprises from about 9.0 to about 10.5 wt % of the porous silica particles.

In particular embodiments, the formulation comprises from about 9.0 to about 10.0 wt % of the porous silica particles, such as about 10.0 wt % or about 9.4 wt % of the porous silica particles.

As described herein, the formulation of the invention comprises a thickening agent, wherein the thickening agent is selected from xanthan gum and microcrystalline cellulose (MCC) or a mixture thereof.

In a particular embodiment, the thickening agent is selected from (i.e. selected from the group consisting of) xanthan gum and microcrystalline cellulose.

As described herein, it has been found that these thickening agents, as opposed to other such agents, allow for the preparation of formulations that provide improved properties in terms of stability and palatability (as a result of, for example, the texture thereof), without inhibiting the biological activity of the silica material.

The skilled person will understand that the microcrystalline cellulose may be colloidal microcrystalline cellulose.

The skilled person will be able to select a suitable amount of each such thickening agent in order to achieve required properties of the formulation, such as the viscosity and flowability thereof.

In particular embodiments, the formulation comprises:
at least about 0.1 wt % (such as at least about 0.2, 0.3, 0.4 or 0.5 wt %) of the thickening agent; and/or (e.g. and)
up to about 1.5 wt % (such as up to about 1.4, 1.3, 1.2 or 1.1 wt %) of the thickening agent.

In particular embodiments, the formulation comprises from about 0.1 wt % to about 1.5 wt % of the thickening agent.

In particular embodiments, the formulation comprises from about 0.2 wt % to about 1.4 wt % of the thickening agent.

In particular embodiments, the formulation comprises from about 0.3 wt % to about 1.3 wt % of the thickening agent.

In particular embodiments, the formulation comprises from about 0.4 wt % to about 1.2 wt % of the thickening agent.

In particular embodiments, the formulation comprises from about 0.5 wt % to about 1.1 wt % of the thickening agent.

In certain embodiments, the thickening agent is microcrystalline cellulose.

In such embodiments (i.e. where the thickening agent is microcrystalline cellulose), the formulation comprises from about 0.8 wt % to about 1.2 wt % of the thickening agent.

In further such embodiments (i.e. where the thickening agent is microcrystalline cellulose), the formulation comprises from about 0.9 wt % to about 1.1 wt % of the thickening agent.

In particular such embodiments (i.e. where the thickening agent is microcrystalline cellulose), the formulation comprises from about 1 wt % of the thickening agent, such as about 1.0 wt % of the thickening agent.

As also described herein, it has been found that the use of xanthan gum as the thickening agent provides a formulation that has yet further improved texture, which results in yet further improved palatability.

In particular embodiments, the thickening agent is xanthan gum.

In such embodiments (i.e. where the thickening agent is xanthan gum), the formulation comprises from about 0.3 wt % to about 0.9 wt % of the thickening agent.

In further such embodiments (i.e. where the thickening agent is xanthan gum), the formulation comprises from about 0.4 wt % to about 0.8 wt % of the thickening agent.

In yet further such embodiments (i.e. where the thickening agent is xanthan gum), the formulation comprises from about 0.5 wt % to about 0.7 wt % of the thickening agent.

In particular such embodiments (i.e. where the thickening agent is xanthan gum), the formulation comprises from about 0.6 wt % of the v, such as about 0.63 wt % of the thickening agent.

The skilled person will understand that the formulation of the invention may comprise further components in order to adjust and/or preserve properties such as the freshness/food safety (i.e. being free from contaminants, such as bacterial and mould growth), taste, smell and/or colour of the formulation.

Thus in particular embodiments, the formulation further comprises:
(d) optionally, one or more preservative, sweetener, flavouring, fragrance and/or colorant.

In particular embodiments, the amount of optional component (d) present in the formulation is up to about 10.0 wt % (such as up to about 9.0, 8.0, 7.0 or 6.0 wt %) of the formulation.

In more particular embodiments, the amount of optional component (d) present in the formulation is up to about 5.0 wt % (such as up to about 4.9, 4.8, 4.7, 4.6, 4.5 or 4.4 wt %) of the formulation.

For example, the formulation may further comprise one or more preservative, such as those food preservative agents suitable for human consumption as known to those skilled in the art.

For example, suitable preservatives will include lactic acid, benzoic acid and pharmaceutically acceptable salts thereof (e.g. sodium benzoate), sorbic acid and pharmaceutically acceptable salts thereof (e.g. potassium sorbate) and citric acid, including mixtures thereof.

Particular preservatives that may be mentioned include citric acid and potassium sorbate, including mixtures thereof.

For the avoidance of doubt, preservatives, such as those described herein, may also be referred to as acidity modifiers.

In a particular embodiment, the formulation further comprises one or more preservatives.

For example, in a particular embodiment, the formulation comprises the preservatives citric acid (e.g. in an amount of about 0.1 to about 0.3 wt % of the formulation, such as about 0.2 wt % of the formulation, e.g. about 0.22 wt % of the formulation) and potassium sorbate (e.g. in an amount of about 0.05 to about 0.15 wt % of the formulation, such as about 0.1 wt % of the formulation, e.g. about 0.09 wt % of the formulation).

The formulation may also comprise one or more flavouring agent and/or sweetener, such as those flavouring agents and sweeteners suitable for human consumption as known to those skilled in the art.

For example, suitable sweeteners may include sugars (such as sucrose, tagatose and allulose), sugar alcohols (such as erythritol, xylitol, sorbitol and mannitol), artificial sweeteners (such as sucralose, saccharin, acesulfame-K and aspartame) and plant extracts (such as steviol glycosides and monk fruit extract). Particular sweeteners that may be mentioned include non-caloric sweeteners, particularly sugar alcohols (such as erythritol) and plant extracts (such as steviol glycosides).

In a particular embodiment, the formulation comprises one or more sweetener.

The skilled person will be able to select a suitable sweetener and amount thereof in order to achieve the required taste profile of the formulation.

In particular embodiments, the sweetener is present in an amount of about 0.01 to about 10.0 wt % of the formulation.

In more particular embodiments, the sweetener is present in an amount of about 0.05 to about 10.0 wt % of the formulation.

In yet more particular embodiments, the sweetener is present in an amount of about 0.1 to about 10.0 wt % of the formulation.

In yet more particular embodiments, the sweetener is present in an amount of about 0.5 to about 10.0 wt % of the formulation.

In yet more particular embodiments, the sweetener is present in an amount of about 1.0 to about 10.0 wt % of the formulation.

In yet more particular embodiments, the sweetener is present in an amount of about 2.0 to about 9.0 wt % of the formulation.

In yet more particular embodiments, the sweetener is present in an amount of about 2.0 to about 8.0 wt % of the formulation.

In yet more particular embodiments, the sweetener is present in an amount of about 2.0 to about 7.0 wt % of the formulation.

In yet more particular embodiments, the sweetener is present in an amount of about 2.0 to about 6.0 wt % of the formulation.

In yet more particular embodiments, the sweetener is present in an amount of about 3.0 to about 5.0 wt % of the formulation.

In yet more particular embodiments, the sweetener is present in an amount of about 3.0 to about 4.0 wt % of the formulation.

In yet more particular embodiments, the sweetener is present in an amount of about 3.5 to about 3.9 wt % of the formulation.

In yet more particular embodiments, the sweetener is present in an amount of about 3.6 to about 3.9 wt % of the formulation.

In particular embodiments, the sweetener is present in an amount of about 4 wt % of the formulation, such as about 3.8 wt % of the formulation.

In a more particular embodiment, the sweetener is erythritol or a mixture of erythritol and steviol glycoside.

In particular such embodiments (i.e. where the sweetener is erythritol and steviol glycoside), the sweetener is present in an amount of about 4 wt % of the formulation, such as about 3.8 wt % of the formulation, e.g. wherein the erythritol is present in an amount of about 3.7 to about 3.8 wt % of the formulation, such as about 3.75 wt % of the formulation, and the steviol glycoside is present in an amount of about 0.01 wt % of the formulation).

As described herein, the formulation may additionally comprise a flavouring (for example, vanilla and/or fruit flavourings), which may derive from an extract and/or artificial agents.

Such flavourings may be present in an amount that is about 0.01 to about 0.50 wt % of the formulation, e.g. about 0.01 to about 0.30 wt % of the formulation, such as about 0.25 wt % of the formulation.

In certain embodiments, the formulation further comprises:
(e) optionally, one or more additional active agent.

As used herein, the term active agent may refer to a component having a biological effect on the subject, which may be referred to as being a biologically active agent.

For example, such additional active agents may include soluble fibres (such as inulin and dextrin), pharmaceutical agents, vitamins, minerals, natural (e.g. plant) extracts, and the like.

In a particular embodiment, the formulation does not contain (or is substantially free of) additional active agents (i.e. active agents other than the silica material of the invention).

For the avoidance of doubt, the formulation may comprise further additional components, which may be selected in order to enhance and/or supplement the properties of the formulation.

For example, the skilled person will understand that other such additional components, such as colorants and fragrances, may be selected in order to provide a formulation that is appealing to the subject. Where present, such components may be present in an amount that is essentially negligible when compared to the other components.

In particular embodiments, the amounts of components (i.e. components (a) to (c), or (a) to (d), or (a) to (e), as required) as specified herein are equal to 100% (in which embodiments, it may be said that the formulation consists of, or consists essentially of, components as specified herein).

In other embodiments, other components may be added to the formulations of the invention, in amounts that may be additional to the components and relative amounts thereof as specified above, without altering the properties of the formulation. In such embodiments, the formulation comprises components (i.e. components (a) to (c), or (a) to (d), or (a) to (e), as required) as specified herein (with relative amounts thereof equal to 100%), optionally comprising further components.

The skilled person will understand that formulations of the invention will contain each component as a mixture thereof. In particular, such mixtures will contain each component such that it is evenly distributed throughout the formulation, which formulations may be described as being homogenous (or substantially homogenous).

As described herein, formulations of the invention will be stable, which may indicate that the composition of the formulation and the distribution of such components therein will not change over an extended period of time, such as a period of at least 42 days (such as at least 1 year (365 days)).

In particular embodiments, the formulation will be stable for at least 42 days (such as at least 1 year (365 days)), such as wherein the formulation will remain homogenous (or substantially homogenous) upon storage for a period of at least 42 days (such as at least 1 year (365 days); e.g. at room temperature and pressure, when shielded from humidity and sunlight).

### Porous Silica Particles

The skilled person will understand that the porous silica particles as provided in the formulations of the first aspect of the invention may be referred to as a plurality thereof, which plurality may be referred to as a porous silica material.

For the avoidance of doubt, porous silica particles having pores in the mesoporous range may also be referred to as mesoporous silica particles, and vice versa.

The skilled person will understand that references herein to pores being of a certain size will refer to the average diameter of the relevant pores (i.e. the average diameter of each individual pore, considering the dimensions thereof). For the avoidance of doubt, the skilled person will understand that references to average pore size may refer in particular to the average size of the opening of each pore (or, in the case of a pore the channel of which internally traverses the body of the particle, the average size of all openings to the pore(s)), which may be referred to as the pore window(s) (or the window(s) of the pore).

For the avoidance of doubt, unless otherwise stated, averages referred to herein will be calculated as the mean average.

Unless otherwise stated, pore sizes as described herein is measured by nitrogen sorption and calculated using the Density Functional Theory (DFT) method (see, for example, the methods as described in Landers, J. et al., Colloids and Surfaces A: Physicochem. Engineering Aspects, 437, 3-32 (2013)). As such, unless otherwise stated, references herein to an average pore size will refer to pore size as measured by nitrogen sorption and calculated using the Density Functional Theory (DFT).

The skilled person will understand that references to the percentage of pores present being in a particular range may be understood to be references to the pore size distribution (PSD) of such particles. As such, references to the percentage of pores present being in a particular range will refer to the combined volume of pores present in each range as a percentage of the total pore volume of the relevant group(s) of pores (e.g. pores in the mesoporous range).

For the avoidance of doubt, references to particles having a particular average pore size may in certain instances include references to pores that are functionally equivalent (e.g. when utilised in the manner described herein) with particles having such average pore sizes.

The skilled person will understand that pore size distribution of the silica material may be measured using DFT pore size distribution curves, which is a technique well-understood by those skilled in the art (see, for example, Olivier, J. P., Conklin, W. B. and Szombathely, M. V., Studies in Surface Science and Catalysis, 87, 81-89 (1994)). The percentage of the pores are calculated from the DFT cumulative pore size distribution curves.

The skilled person will understand that references to porous silica particles having pores in the mesoporous range will take its normal meaning in the art, i.e. as referring to porous silica particles having (or containing/comprising) pores with a diameter in the range 2 to 50 nm, which materials may be referred to as mesoporous and which pores may be referred to as mesopores.

For the avoidance of doubt, the skilled person will understand that the porous silica material referred to in the first aspect of the invention may also have (i.e. further containing/comprising) pores with a diameter outside of the mesoporous range, such as by having micropores (i.e. pores with a diameter of less than 2 nm) and/or macropores (i.e. pores with a diameter of greater than 50 nm).

For the avoidance of doubt, unless otherwise stated, references for percentages of pores as used herein will refer to the percentage by volume.

In a particular embodiment, at least about 40% (i.e. 40% by volume) of the pores present in the silica material of the invention are in the mesoporous range.

In a more particular embodiment, at least about 50%, such as at least about 60%, particularly at least about 70%, of the pores present in the silica material of the invention are in the mesoporous range.

The skilled person will understand that, in relation to the pores in a given range, there may also be calculated an average (i.e. mean average) pore size. As described herein, such average pore size may be measured by the nitrogen sorption technique and calculated using the Density Functional Theory (DFT), which will be well-known to those skilled in the art (see: Olivier, J. P., Conklin, W. B. and Szombathely, M. V., Studies in Surface Science and Catalysis, 87, 81-89 (1994); Landers, 3., et al., Colloids and Surfaces A: Physicochem. Eng. Aspects, 437, 3-32 (2013)). As such, unless otherwise stated, references herein to average pore size will refer to average pore size as measured by nitrogen sorption and calculated using DFT.

In particular embodiments, the average pore size of the pores in the mesoporous range is from about 7.0 to about 24.0 nm.

In more particular embodiments, the average pore size of the pores in the mesoporous range is from about 7.0 to about 23.0 nm.

In yet more particular embodiments, the average pore size of the pores in the mesoporous range is from about 7.0 to about 22.0 nm.

In yet more particular embodiments, the average pore size of the pores in the mesoporous range is from about 7.0 to about 21.0 nm.

In yet more particular embodiments, the average pore size of the pores in the mesoporous range is from about 7.0 to about 20.0 nm.

For example, in certain embodiments the average pore size of the pores in the mesoporous range is:
from about 7.0 to about 19.0 nm;
from about 7.0 to about 18.0 nm;
from about 7.0 to about 17.0 nm;
from about 7.0 to about 16.0 nm;
from about 7.0 to about 15.0 nm;
from about 7.0 to about 14.0 nm;
from about 7.0 to about 13.0 nm; or from about 7.0 to about 12.0 nm.

In certain embodiments, the average pore size of the pores in the mesoporous range is from about 8.0 to about 13.0 nm.

In more particular embodiments, the average pore size of the pores in the mesoporous range is from about 8.0 to about 12.0 nm.

In more particular embodiments, the average pore size of the pores in the mesoporous range is from about 8.0 to about 11.0 nm.

In alternative embodiments, the average pore size of the pores in the mesoporous range is from about 9.0 to about 11.0 nm.

In yet more particular embodiments, the average pore size of the pores in the mesoporous range is from about 9.2 to about 11.0 nm.

In yet more particular embodiments, the average pore size of the pores in the mesoporous range is from about 9.4 to about 10.8 nm.

In yet more particular embodiments, the average pore size of the pores in the mesoporous range is from about 9.5 to about 10.7 nm.

In yet more particular embodiments the average pore size of the pores in the mesoporous range is from about 9.6 to about 10.7 nm.

In yet more particular embodiments, the average pore size of the pores in the mesoporous range is from about 9.5 to about 10.6 nm.

In the most particular embodiments, the average pore size of the pores in the mesoporous range is from about 9.6 to about 10.6 nm.

In further embodiments, the average pore size of the pores in the mesoporous range is from about 8.0 to about 22.0 nm.

In yet further embodiments, the average pore size of the pores in the mesoporous range is from about 9.0 to about 22.0 nm.

In yet further embodiments, the average pore size of the pores in the mesoporous range is from about 10.0 to about 22.0 nm.

In yet further embodiments, the average pore size of the pores in the mesoporous range is from about 10.0 to about 21.0 nm.

In yet further embodiments, the average pore size of the pores in the mesoporous range is from about 10.0 to about 20.0 nm.

In yet further embodiments, the average pore size of the pores in the mesoporous range is from about 9.0 to about 21.0 nm.

In yet further embodiments, the average pore size of the pores in the mesoporous range is from about 10.0 to about 21.0 nm.

In yet further embodiments, the average pore size of the pores in the mesoporous range is from about 11.0 to about 21.0 nm.

In yet further embodiments, the average pore size of the pores in the mesoporous range is from about 12.0 to about 21.0 nm.

In yet further embodiments, the average pore size of the pores in the mesoporous range is from about 13.0 to about 21.0 nm.

In yet further embodiments, the average pore size of the pores in the mesoporous range is from about 14.0 to about 21.0 nm.

In yet further embodiments, the average pore size of the pores in the mesoporous range is from about 15.0 to about 21.0 nm.

In yet further embodiments, the average pore size of the pores in the mesoporous range is from about 16.0 to about 21.0 nm.

In yet further embodiments, the average pore size of the pores in the mesoporous range is from about 16.0 to about 20.0 nm.

In yet further embodiments, the average pore size of the pores in the mesoporous range is from about 17.0 to about 20.0 nm.

In yet further embodiments, the average pore size of the pores in the mesoporous range is from about 17.0 to about 19.0 nm.

In yet further embodiments, the average pore size of the pores in the mesoporous range is from about 17.0 to about 18.0 nm.

In yet further embodiments, the average pore size of the pores in the mesoporous range is about 22 nm (e.g. about 22.0 nm).

The skilled person will understand that, in addition to referring to the (mean) average pore size, as described herein, the silica material of the invention may also be defined by reference to the distribution of pore sizes, such as the distribution of pore sizes of the pores in the mesoporous range.

In particular embodiments of the first aspect of the invention, at least 21% (such as at least at least 22%, at least 23%, at least 24%, at least 25%, at least 26%, at least 27% at least 28% or at least 29%) of the pores in the mesoporous range (by volume) have a diameter in within the range of the specified for the average pore size (i.e. the range as specified for the average pore size).

In more particular embodiments of the first aspect of the invention, at least about 30% of the pores in the mesoporous range have a diameter in within the range of the average pore size.

In yet more particular embodiments of the first aspect of the invention, at least about 35% (such as at least 40% or at least 45%) of the pores in the mesoporous range have a diameter in within the range of the average pore size.

In still more particular embodiments of the first aspect of the invention, at least about 50% (such as at least 55%, at least 60%, at least about 65%, at least about 70% or, particularly, at least about 72%) of the pores in the mesoporous range have a diameter in within the range of the average pore size (i.e. the range given for the average pore size of the pores in the mesoporous range, as defined herein).

For example, in certain embodiments at least about 50% (such as at least 55%, at least 60%, at least about 65%, at least about 70% or, particularly, at least about 72%) of the pores in the mesoporous range have a diameter in within the range about 7.0 to about 25.0 nm.

In certain embodiments, up to about 100% (or up to about 99%, about 95%, or about 90%) of the pores in the mesoporous range have a diameter in within the range of the average pore size.

In certain embodiments, from about 21% to about 100% (or, particularly, about 25% to about 99% or 100%) of the pores in the mesoporous range have a diameter in within the range of the average pore size.

In yet more particular embodiments of the first aspect of the invention, at least about 30% (e.g. about 30% to about 99% or 100%) of the pores in the mesoporous range have a diameter in within the range of the average pore size.

In yet more particular embodiments of the first aspect of the invention, at least about 35% (e.g. about 35% to about 99%) of the pores in the mesoporous range have a diameter in within the range of the average pore size.

In yet more particular embodiments of the first aspect of the invention, about 40% to about 90% (or to about 99% or 100%) of the pores in the mesoporous range have a diameter in within the range of the average pore size.

For example, in a particular embodiment (i.e. a particular embodiment of the first aspect of the invention), at least about 25% (e.g. about 25% to about 99%, such as about 50% to about 99% or 100%) of the pores of the silica particle are mesopores of a size in the range of from about 7.0 to about 25.0 nm (such as about 7.0 to about 18.0 nm, or about 7.0 to about 13.0 nm).

Similarly, in a particular embodiment, at least about 50% (e.g. about 50% to about 99%, such as about 50% to about 90%) of the pores of the silica particle are mesopores of a size in the range of from about 7.0 to about 25.0 nm (such as about 7.0 to about 18.0 nm, or about 7.0 to about 13.0 nm).

In a particular embodiment (i.e. a particular embodiment of the first aspect of the invention), at least about 50% (e.g. about 50% to about 99%) of the pores of the silica particle are mesopores of a size in the range of from about 9.0 to about 12.0 nm.

In a further embodiment, at least about 25% (e.g. at least about 50%, about 60% or about 70%) of the pores of the silica particle are mesopores of a size in the range of from about 9.0 to about 10.2 nm.

In a further embodiment, at least about 25% (e.g. at least about 50%, about 60% or about 70%) of the pores of the silica particle are mesopores of a size in the range of from about 9.0 to about 11.0 nm.

In a more particular embodiment, at least about 25% (e.g. about 25% to about 99%) of the pores of the silica particle are mesopores of a size in the range of from about 9.0 to about 11.0 nm.

In a yet more particular embodiment, at least about 25% (e.g. about 25% to about 99%) of the pores of the silica particle are mesopores of a size in the range of from about 9.0 to about 10.2 nm.

The skilled person will understand that references to porous silica particles having pores in the mesoporous range will necessarily require that such particles are porous, which will include particles behaving in a porous manner. As such, porous silica particles will refer to particles having a significant degree of porosity, which may in certain embodiments be defined by reference to features such as the pore volume and/or surface area of the particles, such as by reference to those parameters as defined herein (which features as described herein may, as with other features described herein, be taken both alone and in combination).

The skilled person will also understand that the total volume of pores in each particle may affect the surface area of the particle.

The skilled person will understand that the surface area of a particle (or a sample of particles) may be calculated using the Brunauer Emmett Teller (BET) theory, a technique well-known to those skilled in the art (see, for example, Brunauer, S., Emmett, P. H., and Teller, E., J. Am. Chem. Soc., 60(2), 309-319 (1938)).

In a particular embodiment, the silica particles have a BET surface area of at least about 150 m²/g.

In a more particular embodiment, the silica particles have a BET surface area of at least about 200 m²/g.

In a yet more particular embodiment, the silica particles have a BET surface area of at least about 300 m²/g (such as at least about 350 m²/g).

In a still more particular embodiment, the silica particles have a BET surface area of at least about 400 m²/g (such as at least about 450 m²/g).

In a particular embodiment, the silica particles have a BET surface area of at least about 500 m²/g.

In particular embodiments, the BET surface area is up to about 1500 m²/g (such as up to about 1200 m²/g or 1000 m²/g).

For example, in a particular embodiment, the silica particles have a BET surface area of from about 200 to about 1500 m²/g.

In a further embodiment, the silica particles have a BET surface area of from about 500 to about 1200 m²/g.

In a yet more particular embodiment, the silica particles have a BET surface area of from about 600 to about 1200 m²/g.

In an alternative embodiment, the silica particles have a BET surface area of from about 600 to about 1000 m²/g.

In a further alternative embodiment, the silica particles have a BET surface area of from about 500 to about 900 m²/g, such as from about 550 to about 900 m²/g.

In a yet further alternative embodiment, the silica particles have a BET surface area of from about 600 to about 850 m²/g.

The skilled person will understand that the porous silica particles may be provided in a variety of shapes.

In a particular embodiment, the silica particles have a substantially non-spherical morphology (i.e. an aspect ratio of greater than 1:1, such as greater than 1.1:1).

In a more particular embodiment, the silica particles have an aspect ratio of greater than 1.5:1, such as greater than 1.8:1.

In a yet more particular embodiment, the silica particles have an aspect ratio equal to or greater than 2:1.

As used herein, the term “aspect ratio” will be understood to refer to the ratio between the largest cross-section diameter of the silica particle and the smallest cross-section diameter.

Alternatively, such particles (i.e. particles having a substantially non-spherical morphology) may be described as having at least one plane (i.e. an equally dividing plane bisecting the particle) of asymmetry (i.e. such that the morphology of the particle differs about the plane).

In a more particular embodiment, the silica particles have an essentially rod-shaped morphology. Thus, in particular embodiments, the porous silica particle may be characterized by having an essentially rod-shaped morphology, as seen by electron microscopy (such as by Scanning Electron Microscopy (SEM) or Transmission Electron Microscopy (TEM), using techniques known to those skilled in the art), such as with a rod-length of from about 0.5 to about 5.0 μm.

As used herein, the term essentially rod-shaped will be understood as referring to a particle of an elongate form resembling a rod, in which the rod may be straight or curved (e.g. such rod shaped particles may be substantially straight).

In an alternative embodiment, the silica particles of the invention may be substantially spherical (or referred to as spherical). Thus, in a particular embodiment, the silica particles of the invention may have an aspect ratio (or an average aspect ratio) of about 1:1.

In further embodiments, the silica particles of the invention may be of amorphous shape.

The skilled person will understand that the term mean particle size, as used herein, will refer to the mean diameter of the particles at the greatest point thereof (e.g. in the case of rod-shaped particles, the length thereof; or in the case spherical particles, the diameter thereof.), which may be measured using techniques well-known to those skilled in the art, for example using electron microscopy techniques (such as by Scanning Electron Microscopy (SEM) or Transmission Electron Microscopy (TEM) technique known to those skilled in the art). In a particular embodiment, particle size is determined using electron microscopy (e.g. using SEM).

In particular embodiments, such as those in which the particles are spherical, the size of particles may be defined by reference to the diameter thereof.

In a particular such embodiment, the silica particles have a mean particle size of from about 0.1 to about 20.0 μm.

In a more particular embodiment, the silica particles have a mean particle size of from about 0.1 to about 15.0 μm.

In a yet more particular embodiment, the silica particles have a mean particle size of from about 0.1 to about 10.0 μm.

In a yet more particular embodiment, the silica particles have a mean particle size of from about 0.5 to about 10.0 μm.

In a still more particular embodiment, the silica particles have a mean particle size of from about 0.5 to about 5.0 μm.

In certain embodiments, the silica particles have a mean particle size of from about 0.5 to about 4.5 μm.

In particular embodiments, the silica particles have a mean particle size of from about 1.0 to about 10.0 μm.

In particular embodiments, the silica particles have a mean particle size of from about 1.0 to about 5.0 μm.

In more particular embodiments, the silica particles have a mean particle size of from about 1.0 to about 4.0 μm.

In more particular embodiments, the silica particles have a mean particle size of from about 1.0 to about 4.0 μm.

In yet more particular embodiments, the silica particles have a mean particle size of from about 2.0 to about 4.0 μm.

In still more particular embodiments, the silica particles have a mean particle size of from about 3.0 to about 4.0 μm.

In further embodiments, such as those in which the particles are rod-shaped, the size of particles may be defined (or also defined) by reference to the width thereof (which will refer to the diameter at the narrowest point).

In a particular such embodiment, the silica particles have a mean width of from about 0.05 to about 0.6 μm.

In a more particular embodiment, the silica particles have a mean width of from about 0.1 to about 0.6 μm.

In a yet more particular embodiment, the silica particles have a mean width of from about 0.1 to about 0.4 μm.

In a yet more particular embodiment, the silica particles have a mean width of from about 0.2 to about 0.4 μm.

The skilled person will understand that porous silica materials of the type described in the present invention are typically non-crystalline. Thus, in certain embodiments, the porous silica particle may be described as a substantially non-crystalline porous silica particle (and materials formed from a plurality of such particles may be described in the same manner).

As such, the porous silica particle may be described as a non-crystalline porous silica particle.

In alternative embodiments, the silica material present in particles as described in the first aspect of the invention may be described as being amorphous. In such embodiments, it will be understood that the term amorphous will indicate that the structure of the silica material (excluding the pores present therein) has no substantial order, such as the order which may be present in a crystalline substance (i.e. the porous silica particles, or silica material, may be referred to as non-crystalline).

As described herein, the skilled person will understand that the silica materials of the invention are porous. As such, silica particles of the invention may be referred to as having a certain minimum total pore volume, as measured using nitrogen sorption (e.g. taken as the volume adsorbed at the highest value of P/P₀, for example, P/P₀=0.995), or a range of such volumes.

In particular embodiments, the total pore volume is at least about 0.2 cm³/g (such as at least about 0.3, 0.4, 0.5, 0.6 or 0.7 cm³/g).

In particular embodiments, the total pore volume is from about 0.2 to about 2.5 cm³/g.

In more particular embodiments, the total pore volume is from about 0.2 to about 2.0 cm³/g.

In yet more particular embodiments, the total pore volume is from about 0.5 to about 1.5 cm³/g.

In still more particular embodiments, the total pore volume is from about 0.6 to about 1.4 cm³/g.

For example, in certain embodiments, the total pore volume is from about 0.7 to about 1.3 cm³/g.

### Medical and Non-Medical Uses

As described herein, the formulation of the invention may be useful in the treatment of certain diseases and disorders, and may therefore be provided in a form suitable for such uses. As such, the formulation of the invention may be described as a pharmaceutical formulation (e.g. an oral pharmaceutical formulation).

In a second aspect of the invention, there is provided a formulation as defined in the first aspect of the invention (including all embodiments and features thereof), for use as a pharmaceutical (or for use in medicine or as a medicament).

In an alternative second aspect of the invention, there is provided the use of a formulation as defined in the first aspect of the invention (including all embodiments and features thereof) as a medical device.

As described herein, development of metabolic disorders, such as type 2 diabetes and obesity, are typically preceded by an increase in certain risk factors that may cause either metabolic or cardiovascular events. Reduction of such risk factors may lead to prevention or delayed onset of the actual disease. Formulations as described in the present invention may be particularly suited for use in the reduction of such risk factors, and in the treatment of resulting conditions (such as obesity, prediabetes, type 2 diabetes and dyslipidaemia).

Moreover, treatment with the formulation of the invention may provide an effective means for the treatment of obesity and reduction of body fat (i.e. body fat in the form of adipose tissue), and therefore may also be suitable for use in the treatment of related conditions.

In a third aspect of the invention, there is provided a method for the treatment or prophylaxis of a metabolic disease or disorder, comprising administering to a patient in need thereof a therapeutically effective amount of a formulation as defined in the first aspect of the invention.

In a fourth aspect of the invention, there is provided a formulation as defined in the first aspect of the invention for use in the treatment or prophylaxis of a metabolic disease or disorder.

In an alternative fourth aspect of the invention, there is provided a formulation as defined in the first aspect of the invention for use in the treatment or prophylaxis of a metabolic disease or disorder.

As used herein, the skilled person will understand that references to a metabolic disease or disorder will refer to diseases or disorders that disrupt normal metabolism, i.e. the process of converting food to energy. As such, such diseases and disorders will include those expected to benefit from adjustment of dietary intake, such as by modulation of the uptake of dietary components (e.g. carbohydrates such as sugars, proteins and lipids).

The skilled person will also understand that metabolic diseases and disorders may in turn give rise to cardiovascular diseases and disorders, which may also be treated (or prophylaxis thereof may occur) as part of the present invention.

In particular embodiments of the fourth aspect of the invention, the treatment or prophylaxis of a metabolic disease or disorder will refer to:
(a) the reduction of metabolic risk-factors of type 2 diabetes;
(b) the treatment or prophylaxis of type 2 diabetes;
(c) the treatment or prophylaxis of prediabetes;
(d) the treatment or prophylaxis of metabolic syndrome;
(e) the treatment or prophylaxis of obesity;
(f) the lowering of, or prevention of increase in, body fat levels in the form of adipose tissue;
(g) the lowering of, or prevention of increase in, triglyceride and/or cholesterol levels; and
(h) the treatment or prophylaxis of dyslipidaemia.

For the avoidance of doubt, the skilled person will understand that references in the fourth aspect of the invention to the lowering of, or prevention of increase in, the levels of certain substances (i.e. the levels of certain substances in the body of the patient) may refer to such lowering in a therapeutic manner, or to the prevention of increase in a prophylactic manner, and as such may be performed in a patient in need thereof.

Alternatively, references in the fourth aspect of the invention to the lowering of, or prevention of increase in, the levels of certain substances (i.e. the levels of certain substances in the body of the patient) may refer to such lowering, or to the prevention of increase, in a non-therapeutic (e.g. cosmetic) manner.

Thus, in a further alternative fourth aspect of the invention, there is provided the use of a formulation as defined in the first aspect of the invention in:
the non-therapeutic lowering of, or prevention of increase in, body fat levels in the form of adipose tissue; and
the non-therapeutic lowering of, or prevention of increase in, triglyceride and/or cholesterol levels.

The skilled person will understand that references to the treatment of a particular condition (or, similarly, to treating that condition) will take their normal meanings in the field of medicine. In particular, the terms may refer to achieving a reduction in the severity and/or frequency of occurrence of one or more clinical symptoms associated with the condition, as adjudged by a physician attending a patient having or being susceptible to such symptoms. For example, in the case of the treatment of type 2 diabetes, the term may refer to achieving a reduction in blood glucose levels experienced by a patient (e.g. postprandial glucose levels, i.e. those experienced following the consumption of food).

As used herein, the term prophylaxis will include references to the prevention of (and, similarly, preventing) the disease or disorder (and vice-versa). As such, references to prevention may also be references to prophylaxis, and vice versa. In particular, such terms may refer to achieving a reduction (for example, at least a 10% reduction, such as at least a 20%, 30% or 40% reduction, e.g. at least a 50% reduction) in the likelihood of the patient (or healthy subject) developing the condition (which may be understood as meaning that the condition of the patient changes such that the patient is diagnosed by a physician as having, e.g. requiring treatment for, the relevant disease or disorder) or experiencing the relevant effect.

Similarly, references to achieving a reduction in risk factors may refer to achieving a clinically significant reduction in the level of at least one of the biomarkers for such risk factors. For example, in certain circumstances, such a reduction may be a reduction of at least 1% (e.g. at least a 2%, 3%, 4%, 5%, 6%, 7%, 8%, 9%, 10%, 15%, 20%, 30% or 40% reduction, such as at least a 50% reduction) of the level by which such risk factors exceed their normal values (i.e. the range of values expected in such a patient in a healthy condition) as known to those skilled in the art.

As used herein, references to a patient (or to patients) will refer to a living subject being treated, including mammalian (e.g. human or animal) patients.

In particular embodiments, references to a patient will refer to human patients.

In alternative embodiments, references to a patient may refer to other mammals, such as livestock (e.g. cattle, pigs, sheep, goats, horses, chickens, turkeys, and the like) and/or household pets (e.g. cats, dogs, rabbits, and the like).

Thus, in certain alternative embodiments, the formulation of the first aspect of the invention may instead be referred to as a veterinary composition.

For the avoidance of doubt, the skilled person will understand that such treatment or prophylaxis will be performed in a patient (or subject) in need thereof. The need of a patient (or subject) for such treatment or prophylaxis may be assessed by those skilled in the art.

As used herein, the terms disease and disorder (and, similarly, the terms condition, illness, medical problem, and the like) may be used interchangeably.

As used herein, the term effective amount will refer to an amount of formulation that confers a therapeutic effect on the treated patient. The effect may be observed in a manner that is objective (i.e. measurable by some test or marker) or subjective (i.e. the subject gives an indication of and/or feels an effect). In particular, the effect may be observed (e.g. measured) in a manner that is objective, using appropriate tests as known to those skilled in the art.

For the avoidance of doubt, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated.

For example, particularly when administered orally to a human patient, the formulation as described in the first aspect of the invention may be administered in doses comprising from about 0.1 to about 20.0 g (e.g. about 0.5 to about 15.0 g, such as from about 1.0 to about 5.0 g, e.g. about 1.0 g, about 2.0 g or about 3.0 g) of the porous silica material, which doses may be administered on one or more occasions daily (i.e. during a 24 hour period).

In particular, the skilled person will understand that doses of the silica material may be administered at suitable intervals, such as intervals corresponding to the consumption of food by the patient (e.g. a meal). As such, the doses referred to herein may be understood to be doses that are timed to be administered with food (i.e. timed to coincide with the times at which the patient consumes food, such as a meal).

As used herein, references to diseases and disorders will be understood by those skilled in the art, such as by references to definitions provided in the international classification of such disorders (see, for example, the International Classification of Diseases (ICD), as provided by the World Health Organisation (WHO), such as that updated as of 1 Jan. 2017).

For example, the disorder referred to as metabolic syndrome, which is sometimes known by other names, may refer to a clustering of at least three of the five following medical conditions: abdominal obesity, high blood pressure, high blood sugar (glucose), high serum triglycerides and low high-density lipoprotein (HDL) levels (as defined by, for example, The National Cholesterol Education Program (NCEP) Adult Treatment Panel III (ATP III)).

As described herein, the formulation of the present invention is able to deliver a therapeutic effect per se. Treatment with such particles and compositions may therefore be therapeutically effective even in the absence of treatment with any other therapeutic agent(s).

In particular embodiments of the fourth aspect of the invention, the method or use does not comprise administration in combination with other therapeutic agents used in the same such method or use (i.e. the formulation of the first aspect of the invention may be used/administered as a mono-therapy).

In more particular embodiments of the fourth aspect of the invention, the method or use does not comprise administration in combination with other therapeutic agents.

In a further embodiment of the fourth aspect of the invention, the method comprises administration of the formulation as defined in the first aspect of the invention as a mono-therapy.

As described herein, the present invention relates to the use of the formulations as described herein as an active therapeutic agent, such as in a method for the reduction of metabolic risk-factors of type 2 diabetes. The present invention also relates to such use in the treatment of prediabetes, which may refer to prophylaxis of prediabetes progressing to a clinical state diagnosed as type 2 diabetes.

For the avoidance of doubt, metabolic risk-factors of type 2 diabetes and methods for measuring the same will be well-known to those skilled in the art. Non-limiting examples of biomarkers for such risk-factors may include: the identification of disturbed (i.e. shifted) levels of low density lipoprotein (LDL), high density lipoprotein (HDL), triglycerides, cholesterol, Apo A1 and/or Apo B; and/or a disturbed ratio between Apo A1 and Apo B, and/or between LDL and HDL (wherein references to “disturbed” levels may refer to a value that is shifted from the normal value for the patient in question by a clinically significant amount, as determined by those skilled in the art); and/or high blood pressure, raised insulin resistance, high glucose levels, raised HbA1c, and any combinations thereof (wherein references to “raised” or “high” levels may refer to a value that is higher than the normal value for the patient in question by a clinically significant amount, as determined by those skilled in the art).

In particular embodiments, the metabolic risk-factors of type 2 diabetes are based on analysis of levels of one or more biomarker selected from the group consisting of: LDL, HDL, triglycerides, cholesterol, Apo A1 and Apo B; the ratio between Apo A1 and Apo B, and between LDL and HDL cholesterol; and blood pressure, insulin resistance and glucose levels, and levels of HbA1c.

In particular embodiments, the metabolic risk-factors of type 2 diabetes may be the identification of elevated levels of HbA1c. Similarly, a patient may be identified as having prediabetes based on the identification of elevated levels of HbA1c in a patient not diagnosed as having diabetes (e.g. type 2 diabetes).

For the avoidance of doubt, those skilled in the art will understand that references to elevated levels of HbA1c will refer to levels above those in the range expected to be observed in such a patient (i.e. a patient of the same type) but in a healthy condition.

For example, according to the American Diabetes Association, HbA1c levels may be identified as being elevated when at or greater than 39.0 mmol/mol (equal to 5.7% DCCT). In particular, a patient may be identified as suffering from prediabetes when having HbA1c levels in the range of from 39.0 mmol/mol to 46.0 mmol/mol (5.7% to 6.4% DCCT; although patients with HbA1c levels greater than 46.0 mmol/mol may be diagnosed as having prediabetes if such patients do not meet the criteria for clinical diagnosis of diabetes, which typically requires HbA1c levels greater than or equal to 47.0 mmol/mol).

The skilled person will also be aware that prediabetes may be diagnosed based on other indicating factors as known to those skilled in the art, such as based on measurement of fasting blood glucose levels (e.g. by identifying a fasting blood glucose of 100-125 mg/dl) or through use of an oral glucose tolerance test (OGTT) (e.g. based on a patient having results from OGTT 2 hour blood glucose of 140 mg/dl-199 mg/dl).

The skilled person will understand that the condition dyslipidaemia may be understood to be a high level of lipids (cholesterol, triglycerides, or both) carried by lipoproteins in the blood. This term may include hypolipoproteinaemia (hyperlipidaemia), which refers to abnormally high levels of total cholesterol, low density lipoprotein (LDL) or triglycerides, as well as an abnormally low level of high density lipoprotein (HDL). Thus, for the avoidance of doubt, dyslipidaemia includes hyperlipidaemia.

As described herein, the formulation of the present invention may act to reduce metabolic risk factors of diabetes in a manner that is independent of the treatment of underlying causative factors, such as obesity.

Thus, in particular embodiments of the fourth aspects of the invention, the:
(a) the reduction of metabolic risk-factors of type 2 diabetes,
(b) the treatment or prophylaxis of type 2 diabetes,
(c) the treatment of prediabetes, and/or (e.g. and)
(d) the treatment or prophylaxis of metabolic syndrome,

is in a non-obese patient (i.e. a patient with a BMI of less than 30), particularly in a non-overweight patient (i.e. a patient with a BMI of less than 25), such as in a patient of healthy body weight (e.g. a patient with a BMI of from 18.5 to 24.9).

As described herein, the formulation of the invention may be useful in medical treatments performed without use of other therapeutic agents (i.e. as a mono-therapy).

In further embodiments of the fourth aspect of the invention, the:
the treatment or prophylaxis of type 2 diabetes;
the treatment of prediabetes;
the treatment or prophylaxis of metabolic syndrome; and/or (e.g. and)
the treatment of dyslipidaemia

is in a patient that is not being administered (i.e. is not taking) another therapeutic agent for the treatment or prophylaxis of such conditions.

In particular embodiments, the reference to cholesterol levels may include references to Apo A1, Apo B and/or non-HDL cholesterol, and/or to LDL cholesterol, and to the LDL/HDL cholesterol ratio.

As described herein, the silica material of the invention may act as a molecular sieve by physically separating smaller molecules from larger molecules through its tailored porosity. This physico-chemical separation leads to a delay and reduction in the digestion of food and uptake of biomolecules into the body, thereby reducing energy uptake, thus lowering and delaying postprandial blood lipid and sugar peaks in animal model systems as well as lowering biomarkers such as HbA1c and blood levels of LDL cholesterol in human subjects.

As used herein, references to lowering of risk factors by reference to particular biological markers may refer to lowering of those risk factors from a raised level to a level closer to (or even within) the range expected in such a patient in a healthy state. For example, in relation to the lowering of HbA1c levels, such a lowering may refer to a lowering of at least 0.1 mmol/mol, such as a lowering of from about 0.1 mmol/mol to about 20.0 mmol/mol. The skilled person will understand that such a lowering may occur following treatment over an extended period of time.

In particular, the formulation of the invention may be used as a pharmaceutical treatment or as an active dietary supplement to increase metabolic health by lowering food efficiency, which may be used in a population of subjects (or patients) at risk of developing prediabetes, or who are already diagnosed with metabolic disease such as, but not limited to, type 2 diabetes, obesity, weight related diseases, abnormal glycaemic homeostasis, and metabolic syndrome.

In a particular embodiment, ingestion of the formulation may affect the total carbohydrate absorption from the digestive system. The term carbohydrate absorption denotes the process occurring in the digestive system involving the break-down of food components, containing complex carbohydrates i.e. sugar polymers longer than a dimer, so that they can be taken up or absorbed by the organism.

In another embodiment, the formulation may, upon use for a prolonged period (e.g. at least one month), affect the levels of HbA1c. The term “HbA1c” denotes glycated haemoglobin and is a laboratory measure of glycaemic control during the preceding 2 to 3 months (Bennett, C. M. et al., Diabet. Med., 24(4), 333-43 (2007)). As the skilled person will know, this biological marker is well-established and used for type 2 diabetes diagnosis and assessment of the risk of complications (Lind, M. et al., Diabetes & Metabolic Syndrome: Clinical Research and Reviews, 2(4), 282-293 (2008)).

Thus, in particular embodiments, the treatment of prediabetes, or the treatment or prevention of diabetes and/or metabolic syndrome, as described herein, may be characterised by (i.e. identified by) the lowering of, or prevention of the increase of, the levels of HbA1c in the patient. Moreover, the methods of treatment of such conditions may be referred to as methods of lowering HbA1c levels in a patient in need thereof.

In particular embodiments, the uses or methods described herein may require that the silica material or composition is administered with food or drink (e.g. with food).

### Use as a Dietary Supplement

As described herein, the formulation of the invention may be useful as a dietary supplement, and may therefore be provided in a form suitable for such uses. As such, the formulation of the invention may be described as a dietary supplement formulation (e.g. an oral dietary supplement formulation).

In a fifth aspect of the invention, there is provided a formulation as defined in the first aspect of the invention (including all embodiments and features thereof) for use as a dietary supplement.

For the avoidance of doubt, formulations referred to as a dietary supplement formulation may also be suitable for use as a pharmaceutical formulation (and therefore referred to as a pharmaceutical formulation), and vice versa. Thus, the formulations of the invention may be referred to as an oral pharmaceutical or dietary supplement formulation, or the like.

The skilled person will understand that formulations of the invention may be taken (i.e. administered) at the same time as, or as part of, food (e.g. a meal) or a drink.

The skilled person will understand that references herein to administration with food (or, similarly, to administration with a meal) or drink may refer to administration at the same time as the food or drink is consumed, or shortly before or after the consumption of food or drink (e.g. up to 2 hours, such as up to 1 hour or, particularly, up to 30 minutes, before or after the consumption of food).

The skilled person will also understand that the formulation of the invention, e.g. when used as a dietary supplement, may induce biological effects as described herein. As such, a dietary supplement formulation as described herein may also be referred to as an active dietary supplement formulation.

The skilled person will understand that administration (i.e. oral administration) of the formulation of the present invention together with food or drink may lower the efficiency thereof, meaning that less energy is taken up from a given amount of nutrients.

Thus, in a sixth aspect of the invention, there is provided a method of lowering the efficiency of a food or drink item, comprising administering with said food or drink item a formulation as defined in the first aspect of the invention (including all embodiments and features thereof).

In an alternative sixth aspect of the invention, there is provided the use of a formulation as defined in the first aspect of the invention in a method of lowering the efficiency of a food or drink item, with said use comprising administering the silica particle or composition with said food or drink item.

In a particular embodiment, the food or drink item is a food item, such as a meal.

In a particular embodiment, the lowering of the efficiency of a food or drink item comprises the lowering of the glycaemic response resulting from consumption of the food or drink item.

In yet another embodiment, the formulation can lower the postprandial carbohydrate uptake into the blood. As used herein, the term postprandial may refer to the levels observed immediately after a meal has been consumed, as measured over time.

In one embodiment, the formulation can upon ingestion simultaneously affect the carbohydrate and lipid absorption from the digestive system. In another embodiment, the particles can upon ingestion affect the levels of blood lipids.

In yet another embodiment, the formulation can, upon ingestion, affect the total postprandial absorption of both carbohydrates and lipids uptake into the blood.

The skilled person will understand that references to the glycaemic response of a food or drink item will take their normal meanings in the art, such as by referring to the plasma glucose levels experienced by a patient immediately following (and therefore attributable to) consumption of the food or drink item, which may be referred to as the post-prandial blood glucose response (change in concentration) elicited when a food or meal that contains carbohydrate is ingested. As such, references to lowering the glycaemic response may refer to the patient experiencing a lower glucose level following consumption of the food or drink item when such consumption occurs with administration of the silica particle or composition than would have been experienced if such consumption had occurred without administration of the silica particle or composition.

Similarly, in a particular embodiment, the lowering of the efficiency of a food or drink item comprises the lowering of plasma triglyceride levels following consumption of the food or drink item.

In such instances, references to lowering of plasma glucose or triglyceride levels may refer to a lowering of at least 1% (such as at least 2%, 3%, 5%, 7%, 10%, 15% or, particularly, at least 20%).

The skilled person will understand that blood plasma levels of particular substances (such as blood glucose levels) may be measured in a patient using techniques well-known to those skilled in the art, such as by routine analysis of a sample of blood taken from the patient at an appropriate time.

### Packaging and Manufacture

The skilled person will appreciate that the formulation of the invention may be packaged (e.g. for distribution and sale) in any suitable manner for a pharmaceutical or food supplement, as appropriate for the intended use.

For example, the formulation of the invention may be packaged in single portion containers, such as containers (e.g. single use bottles or pouches, or stick packs) from which the formulation may be consumed directly.

The formulations of the invention may be prepared using techniques known to those skilled in the art, such as by mixing of the components of the composition (in one process or sequentially in sub-sets thereof) to achieve a substantially homogenous mixture thereof.

Thus, in a further aspect of the invention, there is provided a process of preparing a formulation according to the first aspect of the invention (including all embodiments and features thereof), comprising the step of bringing the components of the formulation together to form a mixture thereof and homogenising said mixture.

Without wishing to be bound by theory, it is thought that the porous silica particles with numerous well-defined pores of confined internal volume, and compositions comprising the same, according to the present invention may function through their ability to act as a molecular sieve and their ability to deliver that function in vivo, such as in the digestive system. This effect is believed to arise through action of the pores present in particles of a certain size. Upon ingestion, the porous silica particles mix with food within the digestive system. In the digestive system, larger food molecules are broken down by digestive enzymes into biomolecules small enough for the body to absorb. The digestive system therefore contains both large and small biomolecules.

It is believed that the silica material of the present invention acts a molecular sieve through its tailored porosity, whereby smaller biomolecules are physically separated from larger biomolecules. Only small molecules will diffuse into the pores of the silica material (i.e. small molecules are molecules that may diffuse into the pores of the silica material whereas large molecules are molecules that may not diffuse into the pores but may interact with the surface of the silica material). This physically separates a fraction of smaller biomolecules such as digestive enzymes and metabolic products, including dietary lipid complexes and carbohydrates, from undigested food in the digestive system. The main digestive enzymes responsible for breaking down sugars and fats are amylases and lipases, respectively. In acting as a molecular sieve, it is believed that these digestive enzymes, among other biomolecules, may enter the pores of the silica through the facilitated diffusion effect arising from the combination of pore sizes as claimed. This physically separates the entrapped enzymes from the undigested food, leading to slower digestion and uptake of nutrients from the digestive system.

It is believed that formulations of the invention allow for the porous silica material to be provided in a form that is stable and easily consumed by a subject, without affecting its function. In particular, such formulations allow for the porous silica material to be presented in a form that does not have the unpleasant mouth feel (i.e. grittiness and dry after-taste) associated with other formulations, thus increasing the palatability of the formulation.

### SUMMARY OF THE FIGURES

FIG. 1: Effect of different formulations on silica amylase adsorption efficacy (A)-(D). Mesoporous silica particles (MSP) suspended in various formulations were tested for their ability to adsorb porcine pancreatic amylase. The dynamic of amylase adsorption by the silica in formulations can be seen in the time course graphs (A-C) and their respective area under the curve (AUC) analysis in (D). Formula 3, Formula 6 and Formula 9 decreased the efficacy of the silica to adsorb amylase by 25%, 40% and 11%, respectively, compared with non-formulated MSP. Data are presented as mean±standard error (SE) (n=2).

### EXAMPLES

The present invention will be further described by reference to the following examples, which are not intended to limit the scope of the invention.

### Example 1: Preparation/Characterization of Mesoporous Silica Particles (MSP) Material

### Materials

Mesoporous silica particles (MSP) were manufactured according to a process previously described (see Baek, J. et al., Nanomedicine 17:1, 9-22 (2022), in particular the experimental procedures described therein, the contents of which are hereby incorporated herein by reference). In brief, a meso-structure templating agent (P123, a triblock copolymer with average molecular weight=5800 g mol⁻¹, PEO₂₀PPO₇₀PEO₂₀) was dissolved in aqueous hydrochloric acid (HCl), with acid concentration equivalent to 1.6 M. Complete dissolution of P123 was followed by addition of tetraethyl orthosilicate (TEOS) under vigorous stirring at 40° C. The final molar ratio of P123: TEOS in the solution was 0.02:1.00 and the molar ratio of TEOS: HCl: H₂O was 1:7:230. The synthesis was kept static at 40° C. for 20 h and further hydrothermally treated for 10 h at 100° C.

### Nitrogen Sorption Analysis

Brunauer-Emmett-Teller (BET) surface area was calculated from sorption isotherm at a relative pressure (p/p°) of <0.2. Total pore volume were recorded at a relative pressure (p/p°)=0.995. Average pore size and pore size distributions were derived from the adsorption curves using the Density Functional Theory (DFT) method. The measurements were performed at liquid nitrogen temperature (−196° C.) using a TriStar II volumetric adsorption analyser and data analysis was performed using the software MicroActive for TriStar II version 2.03 (Micromeritics Instrument Corp., GA, USA).

### Particle Size

Scanning electron microscopy using a JSM-7401F (JEOL Ltd., Tokyo, Japan) was used to characterize the particle size and morphology from SEM micrographs. The mean particle size (length and width) were analyzed from 50 particles using ImageJ (Fiji; see Schindelin J, Arganda-Carreras I, Frise E et al., Fiji: an open-source platform for biological-image analysis., Nat. Methods, 9(7), 676-682 (2012)).

TABLE 1
Material characteristics

| | |
|---|---|
| BET surface area (m²/g) | 925 |
| Total pore volume (cm³/g) | 1.19 |
| Average pore size - all pores ¹ (nm) | 5.2 |
| Average pore size in the mesoporous | 10.2 |
| range - DFT (nm)² | |
| Mean particle size (μm) | 1.1 (length) 0.3 (width) |
| % pores in the mesoporous range ³ | 80 |
| % of mesopores in the 7-25 nm range ⁴ | 73 |
| % of mesopores in the 7-18 nm range ⁵ | 71 |

¹ [4 × Total Pore Volume (cm³/g)/BET Surface Area (m²/g)] × 1000
²Calculated on adsorption curves, using DFT model and assuming a cylindrical pore geometry
³ Calculated from the DFT pore size distribution and total pore volume: [(Pore Volume at 50 nm − Pore Volume at 2 nm)/Total Pore Volume] × 100
⁴ Calculated from the DFT pore size distribution: [(Pore Volume at 25 nm − Pore Volume at 7 nm)/(Pore Volume at 50 nm − Pore Volume at 2 nm) ] × 100
⁵ Calculated from the DFT pore size distribution: [(Pore Volume at 18 nm − Pore Volume at 7 nm)/(Pore Volume at 50 nm − Pore Volume at 2 nm) ] × 100

Various properties of the studied MSP material were measured using above listed techniques with operational conditions. The features identified are described in Table 1 below.

### Example 2: Preparation of Formulations

The following ingredients were purchased from Sigma-Aldrich: potassium sorbate (1.05119.1000), citric acid (27109), Xanthan gum (G1253), Sodium carboxymethylcellulose (419303); Tragacanth gum (G1128), Colloidal microcrystalline cellulose (435244) and guar gum (G4129). Peach flavour was obtained from Einar Willumsen (12101). Erythritol was purchased from Amazon (ASIN B01MFCXDZA). Formulations were homogenized using a Witeg HG-15A homogenizer equipped with the HT1018 dispersing tool.

### Preparation of the Control (Non-Formulated) MSP Suspension

MSP (5 g) were added to 45 g of MilliQ water (MQ H₂O) and homogenized at low speed for 3 min.

### Preparation of Stock Solutions

Solution 1: Potassium sorbate was dissolved in MQ H₂O. Citric acid was then added to the same solution and stirred until dissolved. The concentration of potassium sorbate was 0.125 wt % and the concentration of citric acid was 0.250 wt %.

Solution 2: Peach flavour (0.3 g) was diluted in 3.7 g MQ H₂O.

Formula 1: MSP (5 g) were added to 40.15 g of Solution 1 and 4.75 g of MQ H₂O and homogenized at low speed for 3 min.

Formula 2-10: The thickening agent (concentration according to Table 2; which may also be referred to as a thickener) was slowly added to 40.15 g of Solution 1 and MQ H₂O (amount adjusted to have a total weight of 50 g) while homogenizing. The homogenization speed was slowly increased from low to medium speed as the formulation thickened. Following addition of the thickener, the gels were homogenized at medium speed for another 3 min or until the thickener appeared fully dispersed (lumps no longer visible). Finally, 5 g of MSP were added to the formulation and homogenized at medium speed for 3 min.

Formula 11: Erythritol (2 g) were added to 40.15 g of Solution 1, 0.67 g of Solution 2 and 1.88 g of MQ H₂O. The mixture was stirred until the erythritol was dissolved. Xanthan gum (0.3 g) was slowly added to the solution while homogenizing. The homogenization speed was slowly increased from low to medium speed as the formulation thickened. Following addition of xanthan gum, the formulation was homogenized at medium speed for another 3 min or until the xanthan gum appeared fully dispersed (lumps no longer visible). Finally, 5 g of MSP were added to the formulation and homogenized at medium speed for 3 min.

TABLE 2
Formulations

| | Ingredient concentration (wt %) | | | | | | |
|---|---|---|---|---|---|---|---|
| | | Cit- | Potas- | | | | Peach |
| | | ric | sium | | Thick- | Eryth- | fla- |
| Formula | MSP | acid | sorbate | Water | ener* | ritol | vour |
| Control | 10 | — | — | 90 | — | — | — |
| Formula 1 | 10 | 0.2 | 0.1 | 89.7 | — | — | — |
| Formula 2 | 10 | 0.2 | 0.1 | 89.1 | 0.6 | — | — |
| Formula 3 | 10 | 0.2 | 0.1 | 88.7 | 1 | — | — |
| Formula 4 | 10 | 0.2 | 0.1 | 88.7 | 1 | — | — |
| Formula 5 | 10 | 0.2 | 0.1 | 88.7 | 1 | — | — |
| Formula 6 | 10 | 0.2 | 0.1 | 87.7 | 2 | — | — |
| Formula 7 | 10 | 0.2 | 0.1 | 88.7 | 1 | — | — |
| Formula 8 | 10 | 0.2 | 0.1 | 87.7 | 2 | — | — |
| Formula 9 | 10 | 0.2 | 0.1 | 88.7 | 1 | — | — |
| Formula 10 | 10 | 0.2 | 0.1 | 87.7 | 2 | — | — |
| Formula 11 | 10 | 0.2 | 0.1 | 85 | 0.6 | 4 | 0.1 |

*Xanthan gum in Formulas 2, 3 and 11; Sodium carboxymethylcellulose in Formula 4; Tragacanth gum in Formulas 5 and 6; Colloidal microcrystalline cellulose in Formulas 7 and 8; Guar gum in Formulas 9 and 10

### Example 3: Evaluation of Suspension Stability

The day following manufacture, approximately 20 mL of each formula was transferred to a glass vial and stored in the dark at room temperature. After six weeks (42 days), the samples were observed visually. If separation was observed (clear liquid on top), the suspension was not stable. If no separation was observed after four weeks, the suspension was considered stable under the conditions of this experiment. Results are presented in Table 3.

TABLE 3
Results of suspension stability evaluation

| | | Suspension |
|---|---|---|
| Formula | Thickener | stability |
| Formula 1 | — | Not stable |
| Formula 2 | Xanthan gum 0.6 wt % | Stable |
| Formula 3 | Xanthan gum 1.0 wt % | Stable |
| Formula 4 | Sodium carboxymethylcellulose 1.0 wt % | Not stable |
| Formula 5 | Tragacanth gum 1.0 wt % | Not stable |
| Formula 6 | Tragacanth gum 2.0 wt % | Stable |
| Formula 7 | Colloidal microcrystalline cellulose | Not stable |
| | 1.0 wt % | |
| Formula 8 | Colloidal microcrystalline cellulose | Stable |
| | 2.0 wt % | |
| Formula 9 | Guar gum 1.0 wt % | Not stable |
| Formula 10 | Guar gum 2.0 wt % | not evaluated* |
| Formula 11 | Xanthan gum 0.6 wt % | Stable |

*This formula formed a very thick paste and MSP could not be mixed in uniformly, manufacture was aborted

### Example 4: Evaluation of Suspension Stability after 1 Year Storage at Room Temperature

Some of the samples that appeared stable after six weeks of storage in the dark at room temperature (Example 3) were kept in the same conditions. After 1 year, the samples were observed visually again. If separation was observed (clear liquid on top), the suspension was not stable. If no separation was observed after 1 year, the suspension was considered stable under the conditions of this experiment. Results are presented in Table 4.

TABLE 4
Results of suspension stability evaluation
after 1 year at room temperature

| | | Suspension |
|---|---|---|
| Formula | Thickener | stability |
| Formula 2 | Xanthan gum 0.6 wt % | Stable |
| Formula 3 | Xanthan gum 1.0 wt % | Stable |
| Formula 8 | Colloidal microcrystalline cellulose 2.0 wt % | Stable |
| Formula 11 | Xanthan gum 0.6 wt % | Stable |

### Example 5: Amylase Adsorption Assay

Preparation of working solutions and standard curve samples Prior to the assay, several working solutions were prepared. 2×PBS was prepared by dissolving 1 PBS tablet (Medicago, 09-2052-100) in 100 mL MQ H₂O. Once dissolved, pH was adjusted to 5.4. A working solution of porcine pancreatic amylase (Sigma-Aldrich, A4268) (312 g/mL) was freshly prepared on the day of the experiment by diluting the necessary amount of stock solution with 2×PBS (pH 5.4). A working solution of bicinchoninic acid (BCA) was freshly prepared on the day of the experiment according to the manufacturer's instructions. The amylase standard curve samples (45 μL each) were prepared by serial dilution using 2×PBS (pH 5.4) in 96-well PCR plate (VWR, 732-2387). Following serial dilution, 45 μL of MQ H₂O was added to each standard curve sample. The concentrations of the standard curve samples were: 156, 78, 39, 19.5, 9.8, 4.9, and 0 sg/mL.

### Preparation of Test Formulations

Test formulations were prepared by weighing approximately 1 g of formulation and diluting it in approximately 4 mL of MQ H₂O (1:5 dilution to get 20 mg/mL of silica). Samples were then sonicated to obtain a homogeneous suspension. Briefly, 2 mm microtip (Vibra cell) was fit into the sonicator (Vibra cell) and the silica suspension was sonicated for 3 min at 40% amplitude without pulse.

### Adsorption of Porcine Pancreatic Amylase by Silica

Test formulations were incubated with porcine pancreatic amylase for 15, 30, and 60 minutes at 37° C. for enzyme adsorption/entrapment. Different silica concentrations (1000, 500, 250, and 125 μg/mL, 45 μL each) were prepared by serial dilution using MQ H₂O in the 96-well PCR plate containing the amylase standard curve. 45 μL of porcine pancreatic amylase working solution (312 g/mL; final amylase concentration=156 g/mL) was aliquoted into each well. The plate was sealed (VWR, 391-1254) and incubated for 15, 30 and 60 minutes at 37° C. with vertical rotation using a rotator (Harvard Apparatus, 74-2302). When incubation was completed, the plate was centrifuged at 2000×g for 5 min at room temperature. The supernatant (60 μL) from each well was transferred to a new 96-well plate (Corning, CLS3370).

### Colorimetric Determination of Unbound Porcine Pancreatic Amylase

The amount of unbound porcine pancreatic amylase in the supernatant was determined via BCA assay. Into each well containing the supernatant, 60 μL of BCA working solution was added, the plates were sealed (Bio-Rad, MSB1001) and incubated at 60° C. for 1 hour. The plates were then cooled to room temperature for 15 minutes and the absorbance was read at 562 nm.

### Calculation of the Porcine Pancreatic Amylase Loading Capacity of the Silica

The amount of unbound porcine pancreatic amylase was used to calculate the loading capacity of the silica over time (μg amylase/mg silica). The concentration of unbound amylase in each sample was extrapolated from the slope and the intercept of the amylase standard curve. The amount of amylase adsorbed/entrapped by the silica (in μg/mL) was calculated by subtracting the protein concentration of the unbound amylase from the starting amylase concentration which was 156 μg/mL. Using GraphPad software, the calculated amylase removed (μg/mL) was plotted against the silica concentration (125-1000 μg/mL) for each time point (amylase removal graphs).

The efficacy of MSP was determined by how much amylase (μg) was adsorbed by 1 mg of silica. This was called the loading capacity (μg amylase/mg silica) and was calculated by utilizing the amylase removal graphs. For each time point (15, 30, 60 min), the silica concentration needed to remove approximately 50% of the maximum amylase removal was calculated by utilizing the ‘Nonlinear regression (curve fit)’ and ‘Standard curves to interpolate’ analysis in the GraphPad software. The calculated amount of silica (mg/mL) needed to remove 50% of the amylase (in μg/mL) after 15, 30 and 60 minutes was then used to calculate the loading capacity (μg amylase/mg silica) for each time point. The loading capacity was plotted as a time course graph, where the y-axis is the loading capacity (μg amylase/mg silica) and x-axis is time (15-60 minutes). From this time course graph, the area under the curve (AUC) was calculated for each sample using the analysis tool in GraphPad Prism. The AUC of each sample was then compared to the control non-formulated MSP sample.

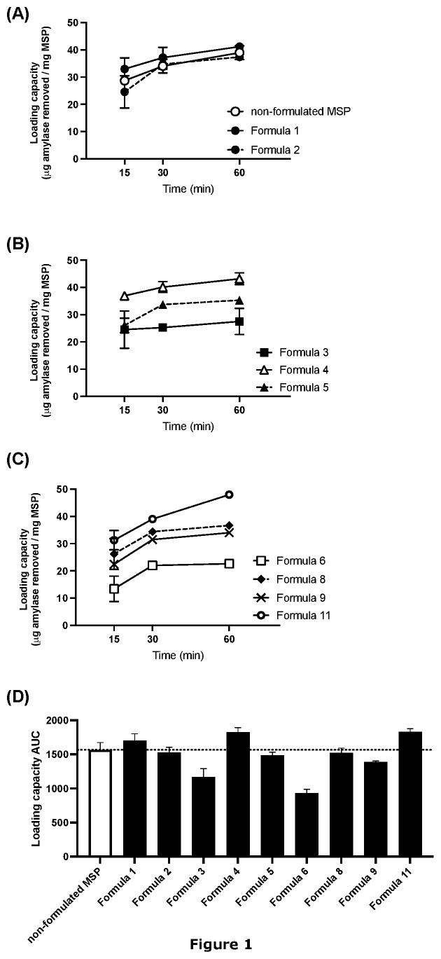

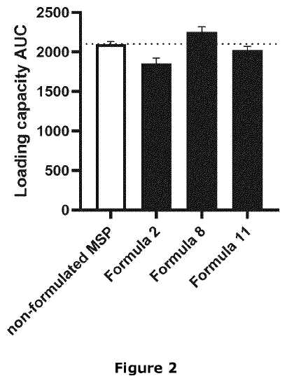

Results are presented in FIG. 1. Formula 3, Formula 6 and Formula 9 decreased the efficacy of the silica to adsorb amylase by 25%, 40% and 11%, respectively, compared with non-formulated MSP. Data are presented as mean±SE (n=2).

### Example 6: Amylase Adsorption Capability after 1 Year Storage at Room Temperature

The samples that appeared stable after six weeks of storage in the dark at room temperature (Example 3) were kept in the same conditions. After 1 year, the samples that did not have a decreased efficacy when compared with non-formulated MSP in the amylase adsorption assay when freshly prepared (Example 5) were analysed again using the method described in Example 5. Results are presented in FIG. 2. All formula tested have acceptable efficacy when compared with non-formulated MSP. Data are presented as mean±SE (n=2).

1. An oral formulation comprising:
(a) water;
(b) porous silica particles having pores in the mesoporous range; and
(c) a thickening agent,
wherein:
the average pore size of the pores in the mesoporous range is from about 7.0 to about 25.0 nm; and
the thickening agent is selected from xanthan gum and microcrystalline cellulose or a mixture thereof.

2. The formulation of claim 1, wherein the formulation comprises from about 70.0 to about 95.0 wt % water.

3. The formulation of claim 1 or claim 2, wherein the formulation comprises from about 5.0 to about 15.0 wt % of the porous silica particles.

4. The formulation of any one of claims 1 to 3, wherein the thickening agent is xanthan gum.

5. The formulation of any one of claims 1 to 4, wherein the formulation comprises from about 0.3 wt % to about 0.9 wt % of the thickening agent.

6. The formulation of any one of claims 1 to 5, wherein the formulation further comprises:
(d) one or more preservative, sweetener, flavouring, fragrance and/or colorant.

7. The formulation of any one of claims 1 to 6, wherein the average pore size of the pores in the mesoporous range is from about 7.0 to about 22.0 nm.

8. The formulation of any one of claims 1 to 7, wherein the average pore size of the pores in the mesoporous range is from about 10.0 to about 21.0 nm.

9. The formulation of any one of claims 1 to 8, wherein the average pore size of the pores in the mesoporous range is from about 10.0 to about 20.0 nm.

10. The formulation of any one of claims 1 to 9, wherein at least about 40% by volume of the pores are in the mesoporous range.

11. The formulation of any one of claims 1 to 10, wherein the silica particles have a BET surface area of at least about 200 m²/g.

12. The formulation of any one of claims 1 to 11, wherein the silica particles have a mean particle size of from about 0.1 to about 20.0 μm.

13. The formulation of any one of claims 1 to 12, wherein the silica particles have a mean particle size of from about 1.0 to about 5.0 μm.

14. The formulation of any one of claims 1 to 13, wherein the silica particles have a total pore volume from about 0.7 to about 1.3 cm³/g.

15. A formulation as claimed in any one of claims 1 to 14 for use as a pharmaceutical.

16. A method for the treatment or prophylaxis of a metabolic disease or disorder, comprising administering to a patient in need thereof a therapeutically effective amount of a formulation as claimed in any one of claims 1 to 14.

17. A formulation as claimed in any one of claims 1 to 14 for use in the treatment or prophylaxis of a metabolic disease or disorder.

18. A method as claimed in claim 16 or composition for use as claimed in claim 17, wherein the treatment or prophylaxis of a metabolic disease or disorder is:
(a) the reduction of metabolic risk-factors of type 2 diabetes;
(b) the treatment or prophylaxis of type 2 diabetes;
(c) the treatment or prophylaxis of prediabetes;
(d) the treatment or prophylaxis of metabolic syndrome;
(e) the treatment or prophylaxis of obesity;
(f) the lowering of, or prevention of increase in, body fat levels in the form of adipose tissue;
(g) the lowering of, or prevention of increase in, triglyceride and/or cholesterol levels; and
(h) the treatment or prophylaxis of dyslipidaemia.

19. A method of lowering the efficiency of a food or drink item, comprising administering with said food or drink item a formulation as claimed in any one of claims 1 to 14.

20. The use of a formulation as claimed in any one of claims 1 to 14 as a dietary supplement.
